(19) United States
(12) Patent Application Publication
(10) Pub. No.: US 2020/0079790 A1
(43) Pub. Date: Mar. 12, 2020

(54) METHODS OF SELECTIVE ARYL- AND HETEROARYL-NITROGEN BOND FORMATION

(71) Applicant: Janssen Sciences Ireland Unlimited Company, Co Cork (IE)

(72) Inventors: Jordan M. Thompson, Redlands, CA (US); John J.M. Wiener, La Jolla, CA (US)

(21) Appl. No.: 16/494,156

(22) PCT Filed: Mar. 15, 2018
(86) PCT No.: PCT/US2018/022569

§ 371 Date: Sep. 13, 2019

Related U.S. Application Data
(60) Provisional application No. 62/471,884, filed on Mar. 15, 2017.

(51) Int. Cl. C07D 495/16; C07D 413/06

(52) U.S. Cl. CPC C07D 495/16; C07D 413/06

(57) ABSTRACT

The disclosure is directed to methods of preparing compounds having the formula (I-A) and compounds involved in the preparation of such compounds.

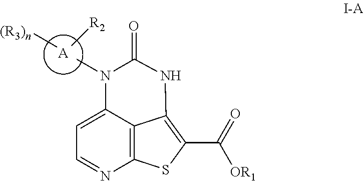

### CROSS-REFERENCE TO RELATED APPLICATIONS

This application claims the benefit of and priority to U.S. Application No. 62/471,884 filed Mar. 15, 2017, the contents of which are incorporated herein by reference in their entireties.

### TECHNICAL FIELD

The disclosure relates to methods of selectively forming aryl-nitrogen bonds and heteroaryl-nitrogen bonds.

### BACKGROUND

Methods of generating aryl-nitrogen and heteroaryl-nitrogen bonds are known. For example, the Buchwald-Hartwig and Ullmann-Goldberg reactions can be used to cross-couple aryl halides with nitrogen moieties. But those reactions are not always selective, especially for compounds that are sterically hindered and/or that have more than one nitrogen moiety available for reaction. Methods of selectively forming aryl-nitrogen and heteroaryl-nitrogen bonds are needed.

### SUMMARY

The present disclosure is directed to methods of preparing compounds of Formula I,

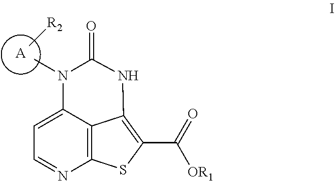

where R₁ is H or C₁₋₆alkyl; A is an optionally further substituted aryl ring or an optionally further substituted heteroaryl ring; and R₂ is selected from the group consisting of C₁₋₆alkyl, C₁₋₆haloalkyl, and halogen. The methods comprise combining a compound of Formula II

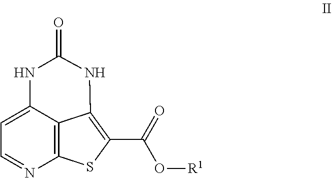

with a compound of Formula III

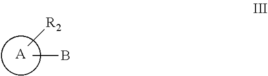

where B is a boronic acid, a boronic ester, or a fluoroborate salt. In certain embodiments, A is pyridyl, phenyl, naphthalenyl, pyrimidinyl, pyrazinyl, pyridazinyl, benzo[d][1,3]dioxolyl, benzothiophenyl or pyrazolyl. In embodiments, A is phenyl or pyridyl. A may be further substituted with one, two, three, or four R₃, as defined herein. In embodiments, R₁ is C₁₋₆alkyl. In embodiments, R₂ is C₁₋₆alkyl. In embodiments, B is —B(OH)₂.

The compounds of Formula II and Formula III may be combined in the presence of a copper salt, a nitrogen-containing ligand, and an organic solvent, for a duration and at a temperature sufficient to produce the compound of Formula I.

In embodiments, the copper salt is a copper halide, copper acetate, copper trifluoromethanesulfonate, or copperhexafluoroantimonate. In embodiments, the copper salt is a copper(II) salt. In embodiments, the copper(II) salt is Cu(II)Cl₂, Cu(II)Br₂, Cu(II)triflate, or Cu(II)(OAc)₂. In embodiments, the copper(II) salt is generated in situ. In embodiments, the copper salt is a copper(I) salt. In embodiments, the copper(I) salt is copper(I)chloride or copper(I)iodide. In embodiments, the amount of the copper salt is a non-catalytic amount. In embodiments, the amount of the copper salt is a catalytic amount.

In embodiments, the nitrogen-containing ligand is a chiral, non-racemic ligand. In embodiments, the nitrogen-containing ligand is selected from the group consisting of tetramethylenediamine, cyclohexane-1,2-diamine, N,N-dimethylcyclohexane-1,2-diamine, N,N,N,N-tetramethylcyclohexane-1,2-diamine, (+)-2,2′-isopropylidenebis[(4R-4-benzyl-2-oxazoline)], (+)-2,2′-isopropylidenebis[(4R-4-phenyl-2-oxazoline)], (+)-2,2′-isopropylidenebis[(4S-4-tert-butyl-2-oxazoline)], (R)-(+)-alpha-methylbenzylamine, (1R,2R)-(+)-1,2-diphenyl-1,2-ethanediamine, 2,6-bis[(4S,5S)-4-methyl-5-phenyl-2-oxazolinyl]pyridine, 2,2′-isopropylidenebis[(4S)-4-tert-butyl-2-oxazoline], 2,2-bis[(4S)-(−)-4-isopropyloxazoline)propane], 2,2′-methylenebis[(4S)-4-tert-butyl-2-oxazoline], (3AS,3′AS,8AR,8′AR)-2,2′-methylenebis[3A,8A-dihydro-8H-indeno[1,2-D]oxazole], (4S)-(+)-4-[4-(tert-butyl)phenyl]-alpha-[(4S)-4-[4-(tert-butyl)phenyl]-2-oxazolidinylidene]-2-oxazolineacetonitrile, (+)-2,2′isopropylidenebis[(4R)-4-phenyl-2-oxazoline], (−)-2,2′-isopropylidenebis[(4S)-4-phenyl-2-oxazoline], 2,2′-methylenebis[(4R,5 S)-4,5-diphenyl-2-oxazoline], 2,6-bis[(3AR,8AS)-(+)-8H-indeno[1,2-D]oxazolin-2-yl]pyridine, (4S)-(+)-phenyl-alpha-[(4S)-phenyloxazolidin-2-ylidene]-2-oxazoline-2-acetonitrile, (+)-2,2′-isopropylidenebis[(4R)-4-benzyl-2-oxazoline, 2,6-bis[(4S)-(−)-isopropyl-2-oxazolin-2-yl]pyridine, 2,6-bis[(4S)-4-[phenyl-2-oxazolinyl]pyridine, 2,2′-methylenebis[(4S)-4-phenyl-2-oxazoline], (4S,4'S)-(−)-2,2′-(3-pentylidene)bis(4-isopropyloxazoline), 2,2′-bis[(4S)-4-benzyl-2-oxazoline], and combinations thereof, or enantiomers thereof. In embodiments, the nitrogen-containing ligand is selected from the group consisting of tetramethylenediamine, cyclohexane-1,2-diamine, N,N-dimethylcyclohexane-1,2-diamine, N,N,N,N-tetramethylcyclohexane-1,2-diamine, and combinations thereof, or enantiomers thereof. In embodiments, the solvent is selected from the group consisting of dimethylformamide, dimethyl acetamide, N-methyl pyrrolidinone, dimethyl sulfoxide, CH₃CN, tetrahydrofuran, dioxane, diethyl ether, ethyl acetate, toluene, benzene, and combinations thereof.

In embodiments, the copper salt is Cu(OTf)₂ and the ligand is

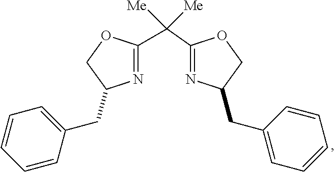 [SMILES: CC(C)(C1=N[C@H](Cc2ccccc2)CO1)C1=N[C@H](Cc2ccccc2)CO1]

or an enantiomer thereof.

In embodiments, the solvent contains less than 5 wt. % of water, based on the weight of the solvent.

In embodiments, the method is carried out in the presence of oxygen.

In embodiments, the compound of Formula I produced by the disclosed methods is a mixture of atropisomers. In embodiments, the mixture of atropisomers consists of an enantiomeric excess (ee) of an atropisomer. In embodiments, the ee of the mixture of atropisomers of the compound of Formula I is at least 1%, preferably at least 10%. In embodiments, the ee of the mixture of atropisomers of the compound of Formula I is 50% or greater. In embodiments, the ee of the mixture of atropisomers of the compound of Formula I is 98% or greater.

The present disclosure is also directed to compounds produced according to the disclosed methods. In embodiments, the atropisomers of the compound of Formula I are

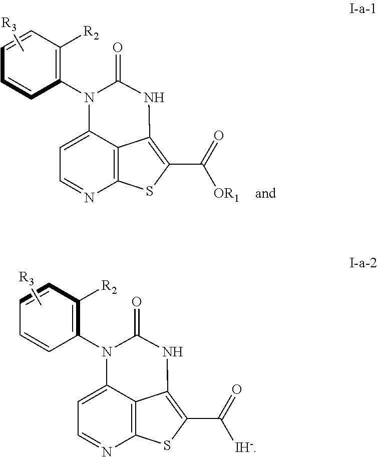

In embodiments, the atropisomers of the compound of Formula I are

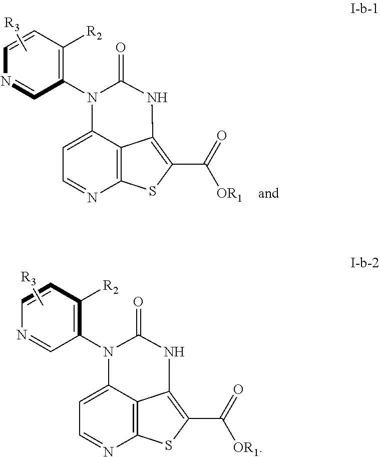

The present disclosure is further directed to compounds of Formula II,

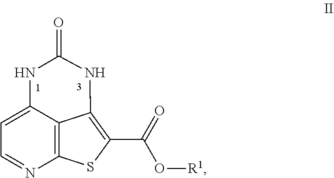

where R₁ is H or C₁₋₆alkyl; or a salt thereof. In embodiments, R₁ is C₁₋₆alkyl.

### DETAILED DESCRIPTION OF ILLUSTRATIVE EMBODIMENTS

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect. Conversely, various features of the disclosed compositions and methods that are, for brevity, described in the context of a single aspect, may also be provided separately or in any subcombination.

### Definitions

The term “alkyl,” when used alone or as part of a substituent group, refers to a straight- or branched-chain alkyl group having from 1 to 12 carbon atoms (“C₁₋₁₂”), preferably 1 to 6 carbons atoms (“C₁₋₆”), in the chain. Examples of alkyl groups include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and groups that in light of the ordinary skill in the art and the teachings provided herein would be considered equivalent to any one of the foregoing examples.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond. In some aspects, the C₁₋₆alk can be substituted with an oxo group or an —OH group.

The term “aryl” refers to carbocylic aromatic groups having from 6 to 10 carbon atoms (“C₆₋₁₀”) such as phenyl, naphthyl, and the like.

The term “heterocycloalkyl” refers to any five to ten membered monocyclic or bicyclic, saturated ring structure containing at least one heteroatom selected from the group consisting of O, N and S. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahyofuranyl, tetrahydropyranyl, piperazinyl, hexahydro-5H-[1,4]dioxino[2,3-c]pyrrolyl, benzo[d][1,3]dioxolyl, and the like.

The term “heteroaryl” refers to a mono- or bicyclic aromatic ring structure including carbon atoms as well as up to four heteroatoms selected from nitrogen, oxygen, and sulfur. Heteroaryl rings can include a total of 5, 6, 9, or 10 ring atoms (“C₅₋₁₀”). Examples of heteroaryl groups include but are not limited to, pyrrolyl, furyl, thienyl, oxazolyl, imidazolyl, pyrazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

The term “halogen” represents chlorine, fluorine, bromine, or iodine. The term “halo” represents chloro, fluoro, bromo, or iodo.

The term “haloalkyl” refers to an alkyl moiety wherein one or more of the hydrogen atoms has been replaced with one or more halogen atoms. One exemplary substituent is fluoro. Preferred haloalkyl groups of the disclosure include trihalogenated alkyl groups such as trifluoromethyl groups.

The term “benzo[d][1,3]dioxolyl” represents the following moiety:

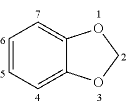

The benzo[d][1,3]dioxolyl moiety can be attached through any one of the 2-, 4-, 5-, 6-, or 7-carbon atoms. In those aspects wherein the “benzo[d][1,3]dioxolyl moiety is substituted with halogen,” the following moieties are preferred:

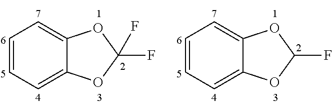

The term “benzothiophenyl” represents the following moiety:

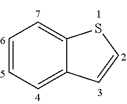

The benzothiophenyl moiety can be attached through any one of the 2-, 3-, 4-, 5-, 6-, or 7-carbon atoms.

The term “phenyl” represents the following moiety:

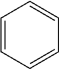

The phenyl moiety can be attached through any of the carbon atoms.

The term “naphthalenyl” (i.e., naphthyl) represents the following moiety:

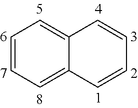

The naphthalenyl moiety can be attached through any one of the 1-, 2-, 3-, 4-, 5-, 6-, 7-, or 8-position carbon atoms.

The term “pyridyl” represents the following moiety:

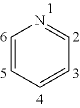

The pyridyl moiety can be attached through any one of the 2-, 3-, 4-, 5-, or 6-position carbon atoms.

The term “pyrimidinyl” represents the following moiety:

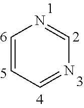

The pyrimidinyl moiety can be attached through any one of the 2-, 4-, 5-, or 6-position carbon atoms.

The term “pyrazinyl” represents the following moiety:

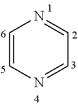

The pyrazinyl moiety can be attached through any one of the 2-, 3-, 5-, or 6-position carbon atoms.

The term “pyridazinyl” represents the following moiety:

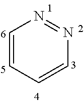

The pyridazinyl moiety can be attached through any one of the 3-, 4-, 5-, or 6-position carbon atoms.

The term “pyrazolyl” represents the following moiety:

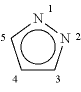

The pyrazolyl moiety can be attached through any one of the 1-, 2-, 3-, 4-, or 5-position carbon atoms.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N,N-diisopropylethylamine, and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

As used herein, the term “isotopic variant” refers to a compound that contains unnatural proportions of isotopes at one or more of the atoms that constitute such compound. For example, an “isotopic variant” of a compound can be radiolabeled, that is, contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that, for example, any hydrogen may be ²H/D, any carbon may be ¹³C, or any nitrogen may be ¹⁵N, and that the presence and placement of such atoms may be determined within the skill of the art. Likewise, the disclosure may include the preparation of isotopic variants with radioisotopes, in the instance, for example, where the resulting compounds may be used for drug and/or substrate tissue distribution studies. Radiolabeled compounds of the disclosure can be used in diagnostic methods such as single-photon emission computed tomography (SPECT). The radioactive isotopes tritium, i.e. ³H, and carbon-14, i.e. ¹⁴C, are particularly useful for their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds of the disclosure, radioactive or not, are intended to be encompassed within the scope of the disclosure.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers.

Stereoisomers that are not mirror images of one another are termed “diastereomers” and those that are non-superimposable mirror images of each other are termed “enantiomers.” An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either an individual enantiomer or as a mixture thereof. A mixture containing equal proportions of the enantiomers is called a “racemic mixture.”

“Atropisomers” refer to stereoisomers that arise because of hindered rotation around a single bond.

The compounds of this disclosure may possess asymmetry; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where an asymmetric (i.e., chiral) center exists in a structure, but no specific stereochemistry is shown for that center, both enantiomers, separately or as a mixture, are encompassed by that structure. The methods for the determination of stereochemistry and the separation of stereoisomers are well-known in the art.

### Methods

Described herein are methods of making compounds of Formula I, or a pharmaceutically acceptable salt or atropisomer thereof.

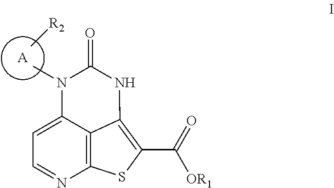

Also described herein are methods of making compounds of Formula I from two compounds requiring the formation of an aryl-nitrogen or heteroaryl-nitrogen bond between the two compounds. Further described herein are methods of making compounds of Formula I from two compounds requiring the formation of an aryl-nitrogen or heteroaryl-nitrogen bond between the two compounds at a site that is sterically hindered at the site of bond formation.

According to the described methods, a compound of Formula I can be produced by combining a compound of Formula II with a compound of Formula III in the presence of a copper salt, a nitrogen-containing ligand, and an organic solvent for a duration and at a temperature sufficient to produce the compound of Formula I, or a pharmaceutically acceptable salt or atropisomer thereof.

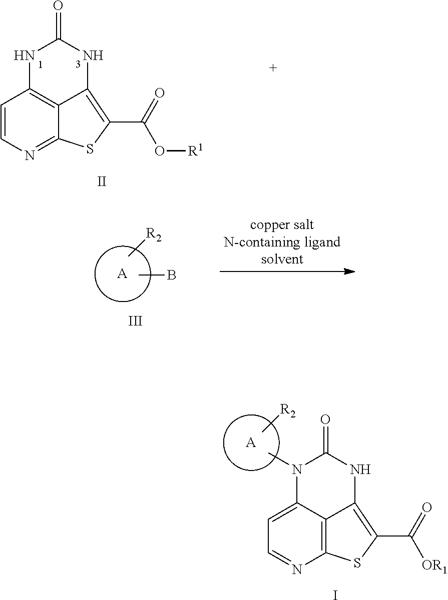

Surprisingly, compounds of Formula I can be produced according to these methods even though the compound of Formula II and the compound of Formula III introduce steric hindrance at the site of bond formation. Steric hindrance of this site occurs, for example, when the A moiety of Formula III is asymmetrical, such as when the A moiety is substituted with a homochiral ligand, or when the R₂ and B moieties on the A moiety are ortho to each other, that is, when R₂ and B are present on adjacent atoms of the A moiety. This steric hindrance can produce atropisomers of compounds of Formula I. The methods of the disclosure will produce compounds of Formula I, preferentially over compounds produced by reactions that occur at the (3) nitrogen of Formula II.

The described methods are surprisingly selective. For example, not only do the described methods selectively couple a compound of Formula II with a compound of Formula III at the (1) nitrogen of Formula II over the (3) nitrogen of Formula II to form a compound of Formula I, but the disclosed methods are also stereoselective, that is, they can preferentially produce one atropisomer of a compound of Formula I over another atropisomer. Accordingly, the described methods may be used to selectively obtain an enantiomeric excess (“ee”) of a desired atropisomer of a compound of Formula I.

The described methods comprise combining a compound of Formula II

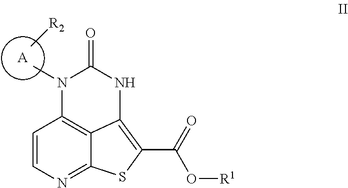

with a compound of Formula III

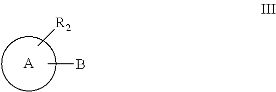

in the presence of a copper salt, a nitrogen-containing ligand, and an organic solvent.

In compounds of Formula II, R¹ can be any moiety that forms a carboxylic acid, an ester or ester derivative such as H or any alky or any aryl. In preferred aspects, R¹ is H or C₁₋₆alkyl. In some embodiments, R¹ is H. In other embodiments, R¹ is C₁₋₆alkyl, for example, methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), or isohexyl (C₆alkyl).

In compounds of Formula III, the A moiety can be an aryl ring or a heteroaryl ring. For example, A can be pyridyl, phenyl, naphthalenyl, pyrimidinyl, pyrazinyl, pyridazinyl, benzo[d][1,3]dioxolyl, benzothiophenyl or pyrazolyl. In certain aspects, A is pyridyl. In some aspects, A is phenyl. In some aspects, A is naphthalenyl. In some aspects, A is pyrimidinyl. In some aspects, A is pyrazinyl. In some aspects, A is pyridazinyl. In some aspects, A is benzo[d][1,3]dioxolyl.

In compounds of Formula III, B is a boron-containing cross-coupling moiety. In preferred aspects, B is —B(OH)₂, a boronic ester, or a fluoroborate salt. In some aspects, B is —B(OH)₂. In other aspects, B is a boronic ester, for example, an alkyl boronic ester or a cycloalkyl boronic ester (e.g., pinacol boronic ester). In other aspects, B is a fluoroborate salt, for example, —BF₄K.

In compounds of Formula III, R₂ can be any substituent that results in steric hindrance of the B moiety or reduces the ability for bond forming reactions to occur at that position. In an embodiment, R₂ is not hydrogen. In an embodiment, R₂ is C₁₋₆alkyl, C₁₋₆haloalkyl, or halogen. In some aspects, R₂ is H. In some aspects, R₂ is C₁₋₆alkyl, C₁₋₆haloalkyl, or halogen. In preferred aspects, R₂ is C₁₋₆alkyl or halogen. In some aspects, R₂ is C₁₋₆alkyl, for example, methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), or isohexyl (C₆alkyl). In other aspects, R₂ is halogen, for example, F, Cl, Br, or I, preferably, F, Cl, or Br.

According to the disclosure, the R₂ and B moieties are arranged ortho to each other on the A moiety of Formula III. In these embodiments, for example, wherein R₂ is C₁₋₆alkyl, C₁₋₆haloalkyl, or halogen, the compounds of Formula I may form atropisomers.

In certain aspects, the A moiety is not further substituted. In certain aspects wherein R₂ is C₁₋₆alkyl, C₁₋₆haloalkyl, or halogen or R₂ is C₁₋₆alkyl or halogen, the A moiety is not further substituted.

In some aspects, A can be substituted with substituents in addition to R² and B. For example, A can be further substituted with 1, 2, 3, or 4 additional substituents. In embodiments, the additional substituent is R³. In embodiments, R³ is independently selected from alkyl, alkoxy, O-aryl, halo, heteroalkyl, cycloalkyl, heterocycloalkyl, aryl, or heteroaryl.

In embodiments, the A moiety is further substituted with at least one R³. In embodiments wherein R₂ is C₁₋₆alkyl, C₁₋₆haloalkyl, or halogen or R₂ is C₁₋₆alkyl or halogen, the A moiety of a compound of Formula III-A may optionally be further substituted with n (wherein n is 1, 2, 3, or 4) number of substituents, R₃, such that a compound of Formula I-A can be produced. In such disclosed methods, combining a compound of Formula II with a compound of Formula III-A in the presence of a copper salt, a nitrogen-containing ligand, and an organic solvent forms a compound of Formula I-A. The following scheme is exemplary of the methods of the disclosure:

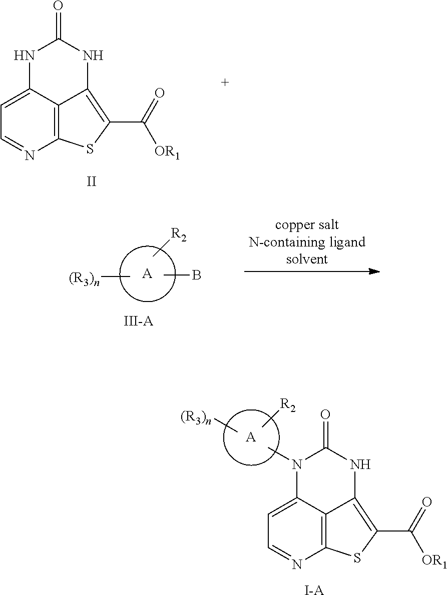

The R₃ substituent(s) is/are independent of R₂ and the B moiety attached to the A moiety. In embodiments, the A moiety is substituted with n number of substituents, R₃. In embodiments, n is one, two or three substituents, R₃. In embodiments, n is one substituent R₃. In embodiments, n is two substituents, R₃. In embodiments, n is three substituents, R₃.

In some embodiments, A is pyridyl, phenyl, naphthalenyl, pyrimidinyl, pyrazinyl, pyridazinyl, benzo[d][1,3]dioxolyl optionally substituted with halogen, benzothiophenyl, or pyrazolyl, wherein the A is optionally substituted with 1 or 2 substituents, R₃. In some embodiments, A is pyridyl, phenyl, naphthalenyl, pyrimidinyl, pyrazinyl, pyridazinyl, benzo[d][1,3]dioxolyl optionally substituted with halogen, benzothiophenyl, or pyrazolyl, wherein the A is substituted with 1 or 2 substituents, R₃.

In some aspects, A is pyridyl optionally substituted with 1 or 2 substituents, R₃.

In some aspects, A is phenyl optionally substituted with 1 or 2 substituents, R₃.

In some aspects, A is naphthalenyl optionally substituted with 1 or 2 substituents, R₃.

In some aspects, A is pyrimidinyl optionally substituted with 1 or 2 substituents, R₃.

In some aspects, A is pyrazinyl optionally substituted with 1 or 2 substituents, R₃.

In some aspects, A is pyridazinyl optionally substituted with 1 or 2 substituents, R₃.

In some aspects, A is benzo[d][1,3]dioxolyl, wherein the benzo[d][1,3]dioxolyl is optionally substituted with 1 or 2 substituents, R₃.

In embodiments, R₁ is C₁₋₆alkyl in compounds of Formula II, and A is phenyl or pyridyl, B is a boronic acid, R₂ is C₁₋₆alkyl, R₃ is C₁₋₆alkyl and n is 1 in compounds of Formula III.

In embodiments, R₁ is C₁₋₆alkyl in compounds of Formula II, and A is phenyl, B is a boronic acid, R₂ is C₁₋₆alkyl, R₃ is C₁₋₆alkyl and n is 1 in compounds of Formula III.

In embodiments, R₁ is C₁₋₆alkyl in compounds of Formula II, and A is pyridyl, B is a boronic acid, R₂ is C₁₋₆alkyl, R₃ is C₁₋₆alkyl and n is 1 in compounds of Formula III.

### Copper Salts

The methods of the disclosure take place in the presence of a copper salt. In some aspects, the copper salt is a copper(II) salt, for example, a copper halide, copper acetate, copper trifluoromethanesulfonate, copper hexafluoroantimonate, or a combination thereof. For example, the copper salt can be Cu(II)Cl₂, Cu(II)Br₂, Cu(II)triflate, Cu(II)(OAc)₂, or a combination thereof. In some aspects, the copper salt is Cu(II)Cl₂. In other aspects, the copper salt is Cu(II)Br₂. In some aspects, the copper salt is Cu(II)triflate. In still other aspects, the copper salt is Cu(II)(OAc)₂.

In those embodiments employing a copper(II) salt, the coupling is preferably conducted under anhydrous conditions. That is, the reaction mixture contains 1 wt % of water or less.

In those embodiments employing a copper(II) salt, the coupling can be carried out in the presence of oxygen. Sources of oxygen include gaseous O₂ and air.

In some embodiments, the copper salt is a copper(I) salt, for example, copper(I)chloride, copper(I)iodide, or a combination thereof. In those embodiments employing a copper(I) salt, the coupling is preferably conducted under anhydrous conditions. That is, the reaction mixture contains 1 wt % of water or less. Preferably, in those aspects employing a copper(I)salt, the coupling reaction is conducted in the presence of an oxygen atmosphere. Sources of oxygen include gaseous O₂ and air.

The copper salts employed in the described coupling reactions can be added to the reaction vessel as a single reagent or mixture of reagents. Alternatively, the copper salt can be generated in situ. For example, in some aspects employing a copper(II) salt, the copper(II) species can be generated in the reaction vessel.

Preferably, the copper salt is present in a stoichiometric amount. That is, the number of moles of the copper salt is equal to, or about equal to, the number of moles of the compound of Formula II. In other embodiments, the number of moles of the copper salt is more than the number of moles of the compound of Formula II. For example, the ratio of the number of moles of the copper salt to the number of moles of the compound of Formula II can be 1:1, about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or about 3:1.

In other aspects, the copper salt is present in less than a stoichiometric amount. That is, the number of moles of the copper salt is less than the number of moles of the compound of Formula II. In some of these aspects, the amount of the copper salt is a catalytic amount. For example, the ratio of the number of moles of the copper salt to the number of moles of the compound of Formula II can be about 0.9:1, 0.8:1, 0.7:1, 0.6:1, 0.5:1, 0.4:1, 0.3:1, 0.2:1, 0.1:1, 0.05:1, 0.04:1, 0.03:1, 0.02:1, 0.01:1, or less.

### Nitrogen-Containing Ligand

The methods of the disclosure require a nitrogen-containing ligand that can bind to, or coordinate with, the copper salt. The nitrogen-containing ligand can be chiral or achiral, but preferred aspects of the disclosure employ a chiral ligand. In those aspects employing a chiral nitrogen-containing ligand, the ligand is provided as a non-racemic mixture. That is, the chiral nitrogen-containing ligand includes an abundance of one stereoisomer over any other stereoisomer.

Nitrogen-containing ligand that can bind to, or coordinate with the copper salt are known in the art. For example, the following ligands can be used in the coupling reactions described herein: tetramethylenediamine, cyclohexane-1,2-diamine, N,N-dimethylcyclohexane-1,2-diamine, N,N,N,N-tetramethylcyclohexane-1,2-diamine, (+)-2,2′-isopropylidenebis[(4R-4-benzyl-2-oxazoline)], (+)-2,2′-isopropylidenebis[(4R-4-phenyl-2-oxazoline)], (+)-2,2′-isopropylidenebis[(4S-4-tert-butyl-2-oxazoline)], (R)-(+)-alpha-methylbenzylamine, (1R,2R)-(+)-1,2-diphenyl-1,2-ethanediamine, 2,6-bis[(4S,5S)-4-methyl-5-phenyl-2-oxazolinyl]pyridine, 2,2′-isopropylidenebis[(4S)-4-tert-butyl-2-oxazoline], 2,2-bis[(4S)-(−)-4-isopropyloxazoline)]propane, 2,2′-methylenebis[(4S)-4-tert-butyl-2-oxazoline], (3AS,3′AS,8AR,8′AR)-2,2′-methylenebis[3A,8A-dihydro-8H-indeno[1,2-D]oxazole], (4S)-(+)-4-[4-(tert-butyl)phenyl]-alpha-[(4S)-4-[4-(tert-butyl)phenyl]-2-oxazolidinylidene]-2-oxazolineacetonitrile, (+)-2,2′isopropylidenebis[(4R)-4-phenyl-2-oxazoline], (−)-2,2′-isopropylidenebis[(4S)-4-phenyl-2-oxazoline], 2,2′-methylenebis[(4R,5 S)-4,5-diphenyl-2-oxazoline], 2,6-bis[(3AR,8AS)-(+)-8H-indeno[1,2-D]oxazolin-2-yl]pyridine, (4S)-(+)-phenyl-alpha-[(4S)-phenyloxazolidin-2-ylidene]-2-oxazoline-2-acetonitrile, (+)-2,2′-isopropylidenebis[(4R)-4-benzyl-2-oxazoline, 2,6-bis[(4S)-(−)-isopropyl-2-oxazolin-2-yl]pyridine, 2,6-bis[(4S)-4-[phenyl-2-oxazolinyl]pyridine, 2,2′-methylenebis[(4S)-4-phenyl-2-oxazoline], (4S,4'S)-(−)-2,2′-(3-pentylidene)bis(4-isopropyloxazoline), 2,2′-bis[(4S)-4-benzyl-2-oxazoline], and combinations thereof.

Chiral, non-racemic ligands include, for example, (+)-2,2′-isopropylidenebis[(4R-4-benzyl-2-oxazoline), (+)-2,2′-isopropylidenebis[(4R-4-phenyl-2-oxazoline), (+)-2,2′-isopropylidenebis[(4S-4-tert-butyl-2-oxazoline), (R)-(+)-alpha-methylbenzylamine, (1R,2R)-(+)-1,2-diphenyl-1,2-ethanediamine, 2,6-bis[(4S,5S)-4-methyl-5-phenyl-2-oxazolinyl]pyridine, 2,2′-isopropylidenebis[(4S)-4-tert-butyl-2-oxazoline], 2,2-bis[(4S)-(−)-4-isopropyloxazoline)]propane, 2,2′-methylenebis[(4S)-4-tert-butyl-2-oxazoline], (3AS,3′AS,8AR,8′AR)-2,2′-methylenebis[3A,8A-dihydro-8H-indeno[1,2-D]oxazole], (4S)-(+)-4-[4-(tert-butyl)phenyl]-alpha-[(4S)-4-[4-(tert-butyl)phenyl]-2-oxazolidinylidene]-2-oxazolineacetonitrile, (+)-2,2′isopropylidenebis[(4R)-4-phenyl-2-oxazoline], (−)-2,2′-isopropylidenebis[(4S)-4-phenyl-2-oxazoline], 2,2′-methylenebis[(4R,5S)-4,5-diphenyl-2-oxazoline], 2,6-bis[(3AR,8AS)-(+)-8H-indeno[1,2-D]oxazolin-2-yl]pyridine, (4S)-(+)-phenyl-alpha-[(4S)-phenyloxazolidin-2-ylidene]-2-oxazoline-2-acetonitrile, (+)-2,2′-isopropylidenebis[(4R)-4-benzyl-2-oxazoline], 2,6-bis[(4S)-(−)-isopropyl-2-oxazolin-2-yl]pyridine, 2,6-bis[(4S)-4-[phenyl-2-oxazolinyl]pyridine, 2,2′-methylenebis[(4S)-4-phenyl-2-oxazoline], (4S,4'S)-(−)-2,2′-(3-pentylidene)bis(4-isopropyloxazoline), 2,2′-bis[(4S)-4-benzyl-2-oxazoline], and combinations thereof.

In some aspects, the nitrogen-containing ligand is tetramethylenediamine, cyclohexane-1,2-diamine, N,N-dimethylcyclohexane-1,2-diamine, N,N,N,N-tetramethylcyclohexane-1,2-diamine, or a combination thereof. In some aspects, the nitrogen-containing ligand is tetramethylenediamine. In other aspects, the nitrogen-containing ligand is cyclohexane-1,2-diamine. In other aspects, the nitrogen-containing ligand is N,N-dimethylcyclohexane-1,2-diamine. In other aspects, the nitrogen-containing ligand is N,N,N,N-tetramethylcyclohexane-1,2-diamine.

Exemplary ligands include, for example,

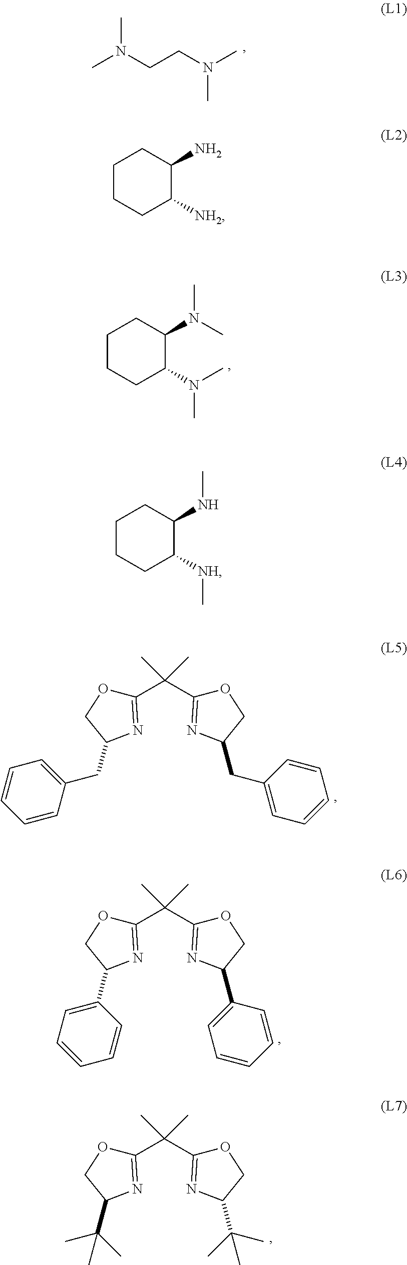

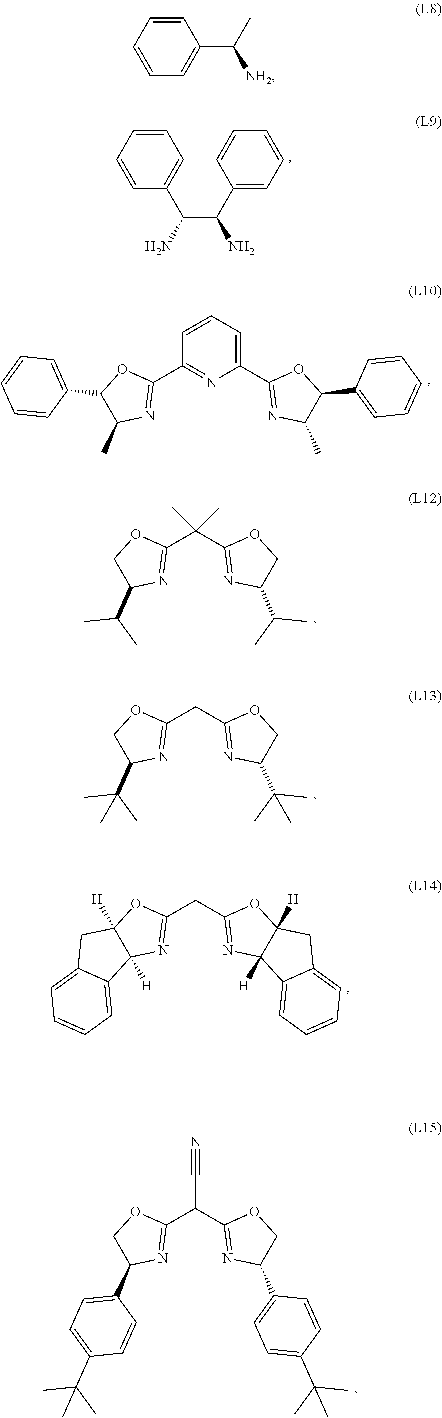

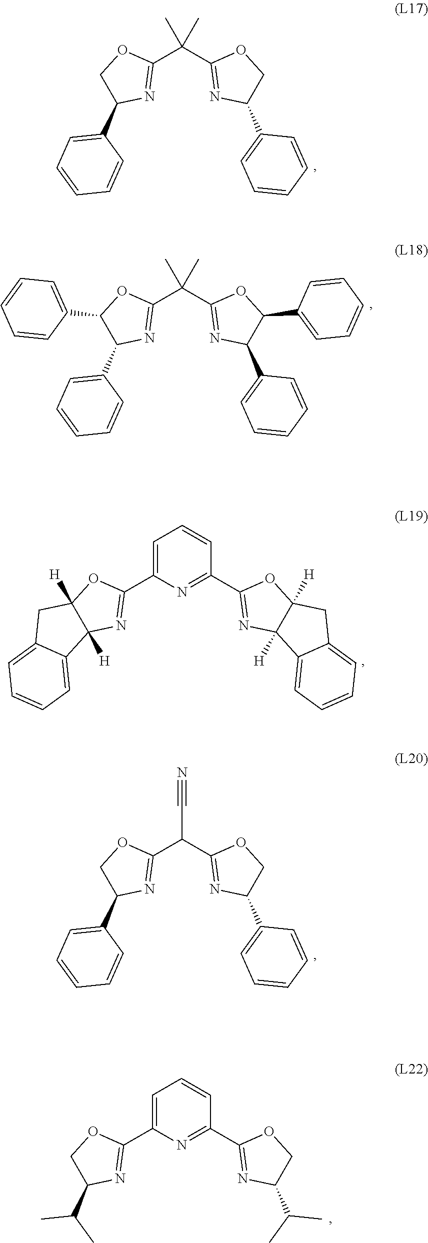 [SMILES: CC(C)(C1=N[C@@H](c2ccccc2)CO1)C1=N[C@@H](c2ccccc2)CO1 ; CC(C)(C1=N[C@H](c2ccccc2)[C@H](c2ccccc2)O1)C1=N[C@H](c2ccccc2)[C@H](c2ccccc2)O1 ; c1cc(C2=N[C@@H]3c4ccccc4C[C@@H]3O2)nc(C2=N[C@@H]3c4ccccc4C[C@@H]3O2)c1 ; N#CC(C1=N[C@@H](c2ccccc2)CO1)C1=N[C@@H](c2ccccc2)CO1 ; CC(C)[C@H]1COC(c2cccc(C3=N[C@@H](C(C)C)CO3)n2)=N1]

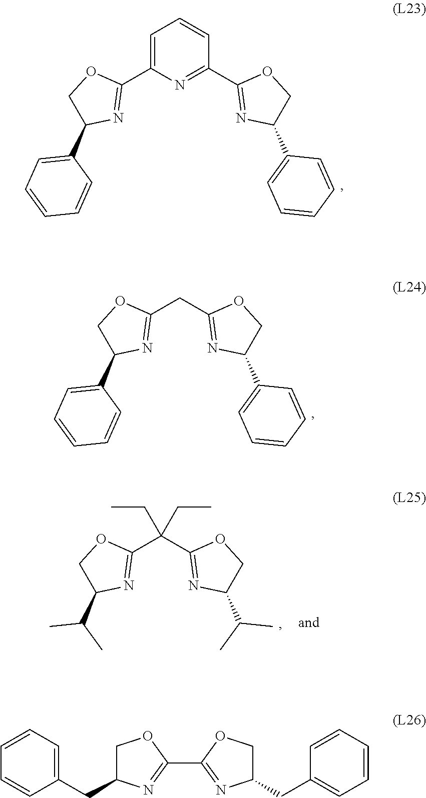 [SMILES: c1ccc([C@H]2COC(c3cccc(C4=N[C@@H](c5ccccc5)CO4)n3)=N2)cc1 ; c1ccc([C@H]2COC(CC3=N[C@@H](c4ccccc4)CO3)=N2)cc1 ; CCC(CC)(C1=N[C@@H](C(C)C)CO1)C1=N[C@@H](C(C)C)CO1 ; c1ccc(C[C@H]2COC(C3=N[C@@H](Cc4ccccc4)CO3)=N2)cc1]

Enantiomers of the nitrogen-containing ligands described herein, for example, L2-L26, are also within the scope of the disclosure.

### Organic Solvent

The methods described herein take place in the presence of one or more organic solvents. Organic solvents suitable for use in the described reactions include dimethylformamide (DMF), dimethyl acetamide (DMA), N-methyl pyrrolidinone (NMP), dimethyl sulfoxide (DMSO), CH₃CN (acetonitrile), tetrahydrofuran (THF), dioxane, diethyl ether, ethyl acetate, toluene, benzene, or a combination thereof. In some aspects, the solvent is DMF, DMA, NMP, DMSO, or a combination thereof. In some aspects, the solvent is acetonitrile. In some aspects, the solvent is THF or diethyl ether. In other aspects, the solvent is ethyl acetate. In some aspects, the solvent is toluene of benzene.

### Temperature

Those of skill in the art will be able to determine an appropriate temperature for carrying out any of the described methods. For example, the methods can be carried out at ambient (i.e., room temperature, 20-25° C.). That is, some methods are carried out without external heating of the reaction mixture. In other aspects, the methods are carried out at elevated temperature, i.e., above room temperature. For example, the methods can be carried out at a temperature of about 30° C. to about 150° C., for example about 30, 35, 40, 45, 50, 55, 60, 65, 70, 75, 80, 85, 90, 95, 100, 105, 110, 115, 120, 125, 130, 135, 140, 145, or about 150° C. Elevated temperatures refer to the internal temperature of the reaction mixture. In other aspects, the methods are carried out at the reflux temperature of the organic solvent.

In other embodiments, the methods can be carried out at a temperature below ambient room temperature. For example, the methods can be carried out at a temperature of about 0° C. to about 15° C., for example, about 0, 5, 10, or about 15° C. In other embodiments, the methods can be carried out at a temperature that is below 0° C., for example, −30, −25, −20, −15, −10, −5, or about 0° C.

### Duration

Those of skill in the art will be able to determine an appropriate time for carrying out any of the described methods. For example, in some aspects, a compound of Formula I is produced in about 30 minutes, 60 minutes, 90 minutes, 120 minutes, or about 150 minutes. In other aspects a compound of Formula I is produced in about 3, 4, 5, 6, 7, 8, 9, 10, 11, 12, 13, 14, 15, 16, 17, 18, 19, 20, 21, 22, 23, or about 24 hours. In other aspects, a compound of Formula I is produced in about 2, 3, 4, 5, 6, or about 7 days.

### Compounds of Formula I

The present disclosure is directed to compounds such as those produced by the disclosed methods. In embodiments, A is phenyl such that the following atropisomers of Formulae I-a-1 and I-a-2 are generated by the disclosed methods. R₁ and R₂ are as described herein. As those of skill in the art will appreciate, R₃ represents 1, 2, 3, or 4 independently selected R₃ substituents as described herein.

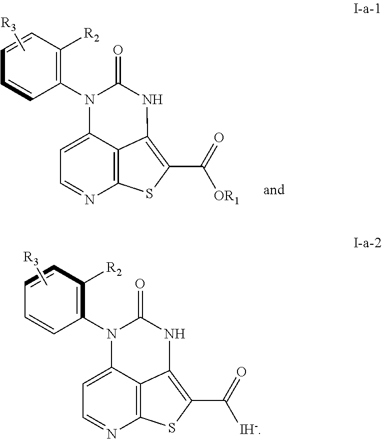

The present disclosure is directed to compounds such as those produced by the disclosed methods. In embodiments, A is phenyl such that the following atropisomers of Formulae I-b-1 and I-b-2 are generated by the disclosed methods. R₁ and R₂ are as described herein. As those of skill in the art will appreciate, R₃ represents 1, 2, 3, or 4 independently selected R₃ substituents as described herein.

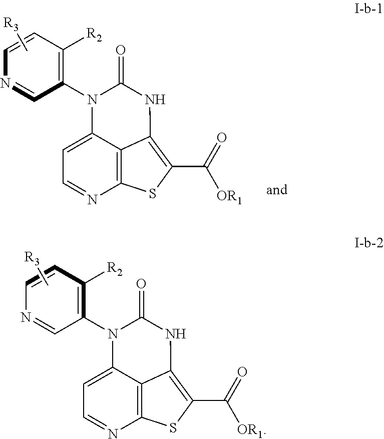

In embodiments, the methods of the present disclosure produce one atropisomer preferentially over the other atropisomer so as to yield one atropisomer in an enantiomeric excess (ee). In some aspects, the compound of Formula I is produced as a mixture of atropisomers wherein the ee is at least 1%. In other aspects, the compound of Formula I is produced as a mixture of atropisomers wherein the ee is at least 10%. In other aspects, the compound of Formula I is produced as a mixture of atropisomers wherein the ee is at least 25%. In other aspects, the compound of Formula I is produced as a mixture of atropisomers wherein the ee is at least 30%. In other aspects, the compound of Formula I is produced as a mixture of atropisomers wherein the ee is at least 35%. In other aspects, the compound of Formula I is produced as a mixture of atropisomers wherein the ee is at least 40%. In other aspects, the compound of Formula I is produced as a mixture of atropisomers wherein the ee is at least 45%. In other aspects, the compound of Formula I is produced as a mixture of atropisomers wherein the ee is at least 50%. In other aspects, the compound of Formula I is produced as a mixture of atropisomers wherein the ee is at least 55%. In other aspects, the compound of Formula I is produced as a mixture of atropisomers wherein the ee is at least 60%. In other aspects, the compound of Formula I is produced as a mixture of atropisomers wherein the ee is at least 65%. In other aspects, the compound of Formula I is produced as a mixture of atropisomers wherein the ee is at least 70%. In other aspects, the compound of Formula I is produced as a mixture of atropisomers wherein the ee is at least 75%. In other aspects, the compound of Formula I is produced as a mixture of atropisomers wherein the ee is at least 80%. In other aspects, the compound of Formula I is produced as a mixture of atropisomers wherein the ee is at least 85%. In other aspects, the compound of Formula I is produced as a mixture of atropisomers wherein the ee is at least 90%. In other aspects, the compound of Formula I is produced as a mixture of atropisomers wherein the ee is at least 95%. In other aspects, the compound of Formula I is produced as a mixture of atropisomers wherein the ee is at least 98%. In other aspects, the compound of Formula I is produced as a mixture of atropisomers wherein the ee is 98% or greater.

Compounds of Formula I are useful as intermediates in the preparation of pharmaceutically active ingredients. For example, compounds of Formula I can be used in the preparation of human bruton's tyrosine kinase (“Btk”) inhibitors. Btk is a ˜76 kDa protein belonging to the Tec family of non-receptor tyrosine kinases. Tec kinases form the second largest family of cytoplasmic tyrosine kinases in mammalian cells, which consists of four other members in addition to Btk: the eponymous kinase TEC, ITK, TXK/RLK and bMX. Tec kinases are evolutionarily conserved throughout vertebrates. They are related to, but structurally distinct from, the larger Src and Syk kinase families. Tec family proteins are abundantly expressed in hematopoietic tissues and play important roles in the growth and differentiation of blood and endothelial cells in mammals.

Based upon Btk expression from IHC studies described in the art, Btk inhibition has the potential to modulate biology associated with b cells, macrophages, mast cells, osteoclasts, and platelet microparticles. Corneth, O. b., et al. Curr. Top. Microbiol. Immunol. Btk Signaling in b Cell Differentiation and Autoimmunity. 2015 Sep. 5. The role of b cells in RA is supported by the therapeutic benefit exhibited in the clinic upon b cell depletion with Rituximab™. Since auto-reactive antibodies play such a critical role in synovial inflammation, therapeutic modulation of the b cell compartment is an attractive mechanism to treat early RA and potentially modulate disease at the earliest stages. b cell depletion in murine models such as collagen-induced arthritis (CIA) prevents arthritis development. Svensson, et al. (1998) b cell-deficient mice do not develop type II collagen-induced arthritis (CIA). Clin Exp Immunol 111, 521-526.

### Examples

Exemplary methods of the disclosure will now be described by reference to the illustrative synthetic scheme for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent.

The methods of the disclosure can be achieved by reference to the following exemplary scheme and procedure:

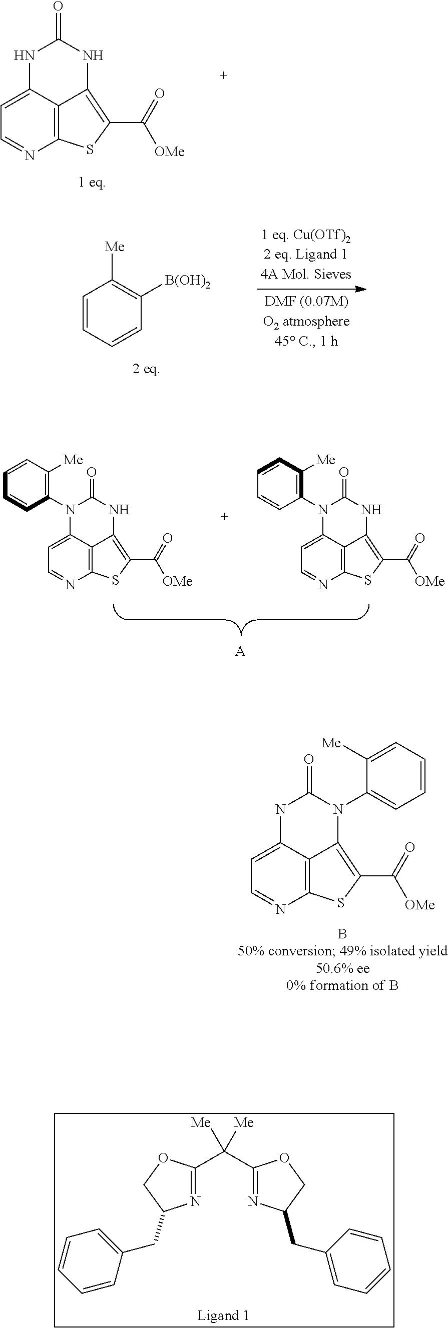

To a 2-dram vial charged with a stir bar, (4R,4′R)-2,2′-(propane-2,2-diyl)bis(4-benzyl-4,5-dyhydrooxazole, ligand 1) (72.713 mg, 0.201 mmol) and copper(II) trifluoromethanesulfonate (36.278 mg, 0.1 mmol) were added with 0.75 mL of DMF and allowed to stir at RT for 3 hours. Then, to a 20 mL amber glass vial charged with a stir bar methyl 4-oxo-4,5-dihydro-3H-1-thia-3,5,8-triazaacenaphthylene-2-carboxylate (25 mg, 0.1 mmol), O-tolylboronic acid (27.273 mg, 0.201 mmol), 4 angstrom molecular sieves (25 mg)(activated by heating under vacuum), the catalyst pre-stirred mixture, and DMF (0.75 mL) were added. The reaction was stirred under an oxygen atmosphere for 1 hour at 45° C. to reveal an absorbance ratio (SM:Products A:by-product b) by LCMS of 1:1:0. The enantiomeric excess of the crude mixture was determined to be 51.2% (HPLC; chiral IA column, 1 mL/min flow rate, 40% EtOH with 0.2% Et₃N: 60% hexanes). K₂CO₃ was added to the mixture, which was extracted three times with ethyl acetate. The combined organic layers were washed three times with brine, dried with K₂SO₄, and then purified by flash chromatography to afford the product (16.8 mg, 49%). The enantiomeric excess of the purified sample was determined to be 50.6% (HPLC) using the same method above.

1. A method of preparing a compound of Formula I, 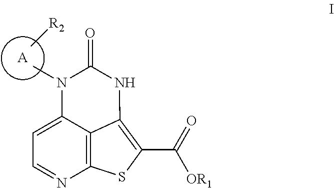
comprising combining a compound of Formula II 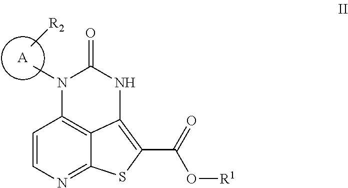
with a compound of Formula III 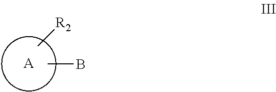
in the presence of a copper salt, a nitrogen-containing ligand, and an organic solvent, for a duration and at a temperature sufficient to produce the compound of Formula I,
wherein
R₁ is H or C₁₋₆alkyl;
A is an optionally substituted aryl ring or an optionally substituted heteroaryl ring;
B is a boronic acid, a boronic ester, or a fluoroborate salt;
R₂ is selected from the group consisting of C₁₋₆alkyl, C₁₋₆haloalkyl, and halogen, and wherein R₂ is at a position that is ortho to B.

2. (canceled)

3. (canceled)

4. The method of claim 3, wherein the compound of Formula I is a mixture of atropisomers and wherein the ee of the mixture of atropisomers of the compound of Formula I is at least 1%

5. The method of claim 4, wherein the ee of the mixture of atropisomers of the compound of Formula I is 98% or greater.

6. The method of claim 1, wherein A is selected from the group consisting of pyridyl, phenyl, naphthalenyl, pyrimidinyl, pyrazinyl, pyridazinyl, benzo[d][1,3]dioxolyl, benzothiophenyl, and pyrazolyl.

7. The method of claim 1, wherein A is further substituted with one, two, three, or four R₃.

8. The method of claim 7, wherein the atropisomers of the compound of Formula I are

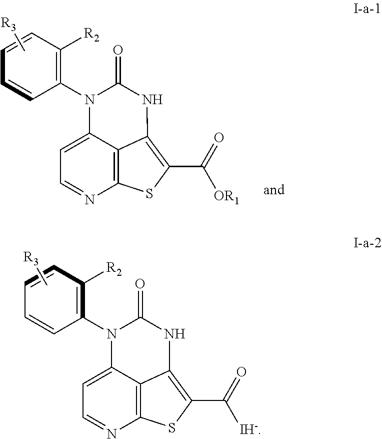

9. The method of claim 7, wherein the atropisomers of the compound of Formula I are

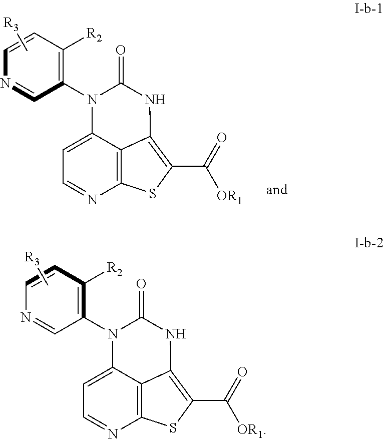

10. The method of claim 1, wherein A is phenyl.

11. The method of claim 1, wherein R₁ is C₁₋₆alkyl.

12. The method of claim 1, wherein R₂ is C₁₋₆alkyl.

13. The method of claim 1, wherein B is —B(OH)₂.

14. The method of claim 1, wherein the copper salt is a copper halide, copper acetate, copper trifluoromethanesulfonate, or copperhexafluoroantimonate.

15. The method of claim 1, wherein the copper salt is a copper(II) salt.

16. The method of claim 15, wherein the copper(II) salt is Cu(II)Cl₂, Cu(II)Br₂, Cu(II)triflate, or Cu(II)(OAc)₂.

17. The method of claim 15, wherein the copper(II) salt is generated in situ.

18. The method of claim 1, wherein the copper salt is a copper(I) salt.

19. The method of claim 18, wherein the copper(I) salt is copper(I)chloride or copper(I)iodide.

20. The method of claim 1, wherein the amount of the copper salt is a non-catalytic amount.

21. The method of claim 1, wherein the amount of the copper salt is a catalytic amount.

22. The method of claim 1, wherein the nitrogen-containing ligand is a chiral, non-racemic ligand.

23. The method of claim 1, wherein the nitrogen-containing ligand is selected from the group consisting of tetramethylenediamine, cyclohexane-1,2-diamine, N,N-dimethylcyclohexane-1,2-diamine, N,N,N,N-tetramethylcyclohexane-1,2-diamine, (+)-2,2′-isopropylidenebis[(4R-4-benzyl-2-oxazoline), (+)-2,2′-isopropylidenebis[(4R-4-phenyl-2-oxazoline), (+)-2,2′-isopropylidenebis[(4 S-4-tert-butyl-2-oxazoline], (R)-(+)-alpha-methylbenzylamine, (1R,2R)-(+)-1,2-diphenyl-1,2-ethanediamine, 2,6-bis[(4S,5 S)-4-methyl-5-phenyl-2-oxazolinyl]pyridine, 2,2′-isopropylidenebis[(4S)-4-tert-butyl-2-oxazoline], 2,2-bis[(4S)-(−)-4-isopropyloxazoline)propane, 2,2′-methylenebis[(4S)-4-tert-butyl-2-oxazoline], (3AS,3′AS,8AR,8′AR)-2,2′-methylenebis[3A,8A-dihydro-8H-indeno[1,2-D]oxazole], (4S)-(+)-4-[4-(tert-butyl)phenyl]-alpha-[(4 S)-4-[4-(tert-butyl)phenyl]-2-oxazolidinylidene]-2-oxazolineacetonitrile, (+)-2,2′isopropylidenebis[(4R)-4-phenyl-2-oxazoline], (−)-2,2′-isopropylidenebis[(4S)-4-phenyl-2-oxazoline], 2,2′-methylenebis[(4R,5 S)-4,5-diphenyl-2-oxazoline], 2,6-bis[(3AR,8AS)-(+)-8H-indeno[1,2-D]oxazolin-2-yl]pyridine, (4 S)-(+)-phenyl-alpha-[(4 S)-phenyloxazolidin-2-ylidene]-2-oxazoline-2-acetonitrile, (+)-2,2′-isopropylidenebis[(4R)-4-benzyl-2-oxazoline, 2,6-bis[(4S)-(−)-isopropyl-2-oxazolin-2-yl]pyridine, 2,6-bis[(4S)-4-[phenyl-2-oxazolinyl]pyridine, 2,2′-methylenebis[(4S)-4-phenyl-2-oxazoline], (4S,4'S)-(−)-2,2′-(3-pentylidene)bis(4-isopropyloxazoline), 2,2′-bis[(4S)-4-benzyl-2-oxazoline, and combinations thereof, or enantiomers thereof.

24. The method of claim 1, wherein the nitrogen-containing ligand is selected from the group consisting of tetramethylenediamine, cyclohexane-1,2-diamine, N,N-dimethylcyclohexane-1,2-diamine, N,N,N,N-tetramethylcyclohexane-1,2-diamine, and combinations thereof, or enantiomers thereof.

25. The method of claim 1, wherein the solvent is selected from the group consisting of dimethylformamide, dimethyl acetamide, N-methyl pyrrolidinone, dimethyl sulfoxide, CH₃CN, tetrahydrofuran, dioxane, diethyl ether, ethyl acetate, toluene, benzene, and combinations thereof.

26. The method of claim 1, wherein the copper salt is Cu(OTf)₂ and the ligand is 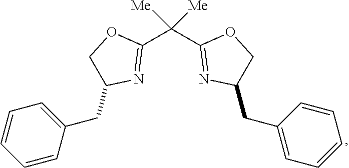 [SMILES: CC(C)(C1=N[C@H](Cc2ccccc2)CO1)C1=N[C@H](Cc2ccccc2)CO1]
or an enantiomer thereof.

27. The method of claim 1, wherein solvent contains less than 5 wt. % of water, based on the weight of the solvent.

28. The method of claim 1, wherein the method is carried out in the presence of oxygen.

29. (canceled)

30. A compound of Formula II, 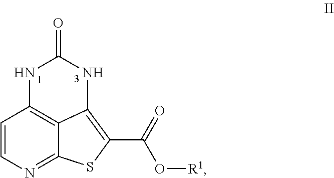
wherein R₁ is H or C₁₋₆alkyl;
or a salt thereof.

31. The compound of claim 30, wherein R₁ is C₁₋₆alkyl.
